(19) United States
(12) Patent Application Publication
(10) Pub. No.: US 2012/0029318 A1
(43) Pub. Date: Feb. 2, 2012

(54) SPONTANEOUS EXERCISE ELECTROCARDIOGRAM SYSTEM

(75) Inventors: Bo-Jau KUO, Taipei (TW); Ching-Hsiu YANG, Taipei (TW)

(73) Assignee: NATIONAL YANG MING UNIVERSITY, Taipei (TW)

(21) Appl. No.: 12/905,812

(22) Filed: Oct. 15, 2010

(30) Foreign Application Priority Data
Jul. 27, 2010 (TW) 099124778

(51) Int. Cl. A61B 5/0205

(52) U.S. Cl. 600/301

(57) ABSTRACT

Disclosed is a spontaneous exercise electrocardiogram system, adapted to synchronously record and analyze a user to obtain a spontaneous exercise electrocardiogram. The system comprises a electrocardiogram device and a analyzing computer. The electrocardiogram device comprises: an electrocardiogram sensor and a 3-dimensional acceleration sensor for detecting the user's electrocardiogram signals and acceleration signals, respectively. Upon the analyzing computer receives the information from the electrocardiogram device, an indicator of physical activity (PA) or new physical activity (NPA) can be determined. After the heart rate of electrocardiogram and heart rate variability being matched and analyzed, the user's spontaneous exercise electrocardiogram can be established.

### BACKGROUND OF THE INVENTION

1. Field of the Invention

The present invention relates to a physiology monitoring system, and particularly relates to a spontaneous exercise electrocardiogram system.

2. Prior Art

Traditional exercise electrocardiogram devices (e.g. a treadmill or stationary bike) can be used for diagnosis, treatment effect evaluation of cardiac surgery, and prognosis for some specific diseases (e.g. slight coronary artery disease). Due to stress on the heart increases during exercise, the exercise electrocardiogram is suitable for assessment for patients. However, it might have a risk that myocardial ischemia occurs, so that a physician should participate simultaneously in case of treatment is needed. The exercise electrocardiogram has different categories due to different requirements, and it usually takes 10-15 minutes.

Although the traditional exercise electrocardiogram takes a short period, cardiac vascular diseases of the patient may not be detected during the transient process. While an electrocardiogram exercise carries out, the physician needs to supervise the whole process due to its risk. On the other hand, for a patient who may have the cardiac vascular disease, in such a way of increasing loading on the heart may induce severe diseases. Thus, researchers continually concern if there is a better way, which can be taken so that the patient can be monitored in the daily life (with various physical activity), to replace the traditional method. If there is a device, which can monitor the patient occurring cardiac abnormality with high or low activity, and measure its heart rate and ECG instantly. The device not only meets the patient's requirement, but also can be utilized for long-term monitoring. It dose not make the patient under a huge pressure and consume manpower a lot. The device should be a wonderful tool for patients or people who might have cardiac vascular disease.

While a myocardial infarction or heart attack occurs, the abnormal ECG is shown. However, the ECG is shown normal while people do not break out disorders or in rest. Therefore, an exercise electrocardiogram (cardiac exercise test) can be used to detect if a person having disorder. In hospital, the treadmill is often used for the purpose. The patient walks on the treadmill with increasing speed in different phase, and the heart loading and its oxygen requirement also accordingly increases. Once heart muscle occur an oxygen shortage, and the ECG instantly shows abnormality.

The heart attack, such as arrhythmia, is usually paroxysmal, and it mostly occurs without any induction or warning. For many patients, their acute myocardial infarction or sudden death takers place at the first time and also the last time. Some symptoms in cardiac diseases, such as chest pain or heart failure caused by hypoxia, not occur in the rest time, but happen after the vigorous exercise or working. On the other hand, the abnormal ECG is unable to completely predict whether the patient with acute exacerbation of heart attack, and it is not to mention when will happen. The traditional health examination has a few of categories relating to cardiac examination, and most of them are to carry out in rest or in a static state. It is insufficient for cardiac risk assessment yet.

The patient who is suitable for traditional exercise electrocardiogram includes conditions as follow: (1) The patient with assisted diagnosis of coronary artery disease. (2) The patient with assessment for coronary artery disease or prognosis of myocardial infarction. (3) The patient with treatment efficiency evaluation after taking Percutaneous Transluminal Coronary Angioplasty (PTCA) or coronary artery bypass surgery. (4) The patient having arrhythmia, assessed for the severity relating to exercise. (5) The patient with congenital heart disease, assessed for exercise tolerance. (6) The patient taken pacemaker implantation, assessed for the pacemaker function. (7) The patient having heart disease or hypertension, with assessment for exercise tolerance after treatment.

The examination includes the steps as described below. According to the standard exercise electrocardiogram process, amount of exercise gradually increases. There are totally seven levels (the maximum amount is in level seven), 3 minutes for each level and blood pressure, heart rate and ECG information is recorded respectively. Equipment for examination includes Ergometer (for upper limb), treadmills or stationary bikes, which are capable of obtaining the exercise electrocardiogram while heart is in oxygen shortage. It takes about 15-20 minutes. A physician and a technician should be arranged to accompany with the examination.

Exercise ECG abnormality includes: (1) ST segment depression; (2) ST segment elevation; (3) Amplitude variation of R wave: If R wave of the patient continually increases, which may suggest a myocardial ischemia; (4) Arrhythmia.

The exercise electrocardiogram usually takes 15-20 minutes, which is better than traditional electrocardiogram. However, there are some problems including: (1) The cause of disease may not easy to be identified due to the short examination time. (2) The examination consumes manpower. (3) To increase a patient's cardiac loading may induce the heart disease.

### Physical Activity (PA) Monitoring

In the prior art, usually a predetermined threshold is set while measuring acceleration for a person by an acceleration sensor. There are different acceleration variations occurred in movements. When an acceleration variation exceeds the predetermined threshold (e.g. 0.025 g), it is recorded as a movement. During a predetermined period, the counts of movements are accumulated to represent the physical activity (PA) of the person (unit: count/minute), and this is a standard method at the present publication and even taken by most of products. However, there is an inevitable drawback that results of measuring physical activity by thresholds are in all-or-none principle and the exact amount of physical activity can not be measured. The activity with small amplitude and higher counts will be amplified, and the activity with huge amplitude and lower counts will be minified. This method is frequently used for energy consumption, but has never been used in exercise electrocardiogram. The present invention is to apply the PA with a novel algorithm to the exercise electrocardiogram.

### HRV Analysis

The autonomic nervous system, classically divided into two subsystems: the parasympathetic nervous system and sympathetic nervous system. The autonomic nervous system controls several important conscious and unconscious activities in human, such as heart rate, blood pressure, bronchial resistance, sweating and metabolism. In recent years, plenty of new technologies to evaluate the autonomic functions were successfully developed. Given the sophisticated computer hardware and spectrum analysis technology available, today it is possible to detect and perform quantitative analysis of a person autonomic cardiac activity in light of the minute fluctuations of heart rate, known as heart rate variability (HRV), taken while the person is at rest. In other words, the new technologies allow a user to analyze or evaluate a normal person's autonomic functions without interfering with the person's daily life.

HRV can be analyzed by ways of Standard Deviation of Normal to Normal Intervals (SDNN) or spectrum analysis. With spectrum analysis, researchers discovered that the minute fluctuations of heart rate variability can be definitely divided into two groups, that is, high-frequency (HF) component and low frequency (LF) component. The HF component is synchronous to animals breath signals, so it is also known as breath component, which occurs approximately every three seconds in a human being. The source of the LF component that takes place approximately every ten seconds in a human being remains unidentified, though researchers infer that they are relevant to vascular motion or baroreflex. Some academics went further to divide the LF component into two categories, that is, very low frequency (VHF) component and low frequency component.

Many physiologists and cardiologists believe that the HF component or total power (TP) reflects parasympathetic functions, whereas the ratio of LF component to HF component (LF/HF) reflects sympathetic activity. It is identified that HRV reflects many physiological functions in the previous study. According to Framingham Company's investigation, the death rate of an elder whose LF component of heart rate variability decreases by a standard deviation is 1.7 times that of normal persons and the LF component of heart rate variability vanishes in a brain-dead person. Furthermore, there are changes in heart rate variability in a patient who exhibits rejection reactions after heart transplantation. During an operation, heart rate variability reflects depth of anesthesia. Gender and age certainly determine sympathetic functions and parasympathetic functions. For example, sympathetic functions and parasympathetic functions are active in young persons, but rather inactive in old persons; in males, sympathetic functions prevail but parasympathetic functions yield; conversely, parasympathetic functions excel sympathetic functions in females. Afterward, the fact that women's sympathetic functions increase during pregnancy, is found in hospitals, but any overreaction may be complicated by, or even contribute to, life-threatening preeclampsia.

If HRV can be applied to spontaneous exercise electrocardiogram, many physical indicators of the user can be obtained. However, all the present products have not applied this technology yet.

At present, miniaturized physical signal collecting apparatus is still implemented with the traditional way. However, the accelerator developed in recent years is designed with miniaturized size, light weight and easy operation. But whose analysis is based on the relation analysis between the acceleration variation of the body and the heart rate. This method is associated with calculating the physical activity and energy consumption, and not utilizes the exercise electrocardiogram abnormality. The present invention bases on the principle of physical activity, and further applies for analysis of traditional exercise electrocardiogram. This not only alleviates the patient's pressure, but also broadens the scope to the common health examination

### SUMMARY OF THE INVENTION

It is an object of the present invention to provide a spontaneous exercise electrocardiogram system.

In one embodiment of the present invention, the spontaneous exercise electrocardiogram system comprises: an electrocardiogram device, comprising an electrocardiogram sensor for capturing and recording an electrocardiogram signal of the user to obtain an electrocardiogram (ECG) data, and a 3-dimensional acceleration sensor for capturing an acceleration signal of the user to obtain an acceleration variation; and an analyzing computer, adapted to receive the ECG data from the electrocardiogram device to obtain the heart rate and heart rate variability of the user; and to receive the user's acceleration signal to determine a physical indicator of the user, wherein upon the user's heart rate and heart rate variability are matched and analyzed, the user's spontaneous exercise electrocardiogram can be established.

Preferably, the electrocardiogram device is a neck-worn type, patch type, button type, wrist-worn type, or belt type 12-lead electrocardiogram device.

Preferably, the electrocardiogram device further comprises a wireless transmission module adapted to transmit the signal of electrocardiogram sensor and 3-dimensional acceleration sensor to the analyzing computer.

Preferably, the physical indicator is a PA (physical activity), NPA (new physical activity) or an indicator derived from the acceleration signal of the user.

Preferably, the cardiac abnormality comprises abnormal PQRST waves, heart rate and heart rate variability of electrocardiogram.

Preferably, the instantaneous physical indicator of the user is automatically obtained by the analyzing computer and matched with the instantaneous electrocardiogram.

Preferably, the components of x-axis, y-axis and z-axis at each time-point of the user is sensed and cumulated to obtain the total acceleration value for each time-point, and the total acceleration value is calculated to obtain its root mean square (RMS) of acceleration variation to be the acceleration variation of the user.

In the present invention, as integrated with the miniaturized physical signal collecting apparatus, simultaneous recording technology, electrocardiogram signal and 3-axis acceleration signal detecting technology, and analytic algorithm, so that the traditional treadmill can be replaced. Users can keep their routine life normally without doing some specific movements at particular place (i.e. hospital). While the accumulated data is sufficient, relation between physical activity and abnormal ECG can be established, and the spontaneous exercise electrocardiogram is accordingly obtained. Not only can the advantages of traditional exercise electrocardiogram be retained, but also the patient avoids being with high pressure or has the adverse effect that examination time may be too short. The system of the present invention is suitable for healthy people, patients having heart disease, people with high pressure, the general elder, or patients having other diseases, such as the cardiovascular disease or the metabolic disease. Thus, the system can be applied on health care, disease diagnose, and severe disease monitoring.

### BRIEF DESCRIPTION OF THE DRAWINGS

FIG. 1 is a block diagram of spontaneous exercise electrocardiogram system of the present invention;

FIG. 2 illustrates a belt type electrocardiogram device; and

FIG. 3 illustrates relations between abnormal ECG and physical activity.

### DETAILED DESCRIPTION OF THE INVENTION

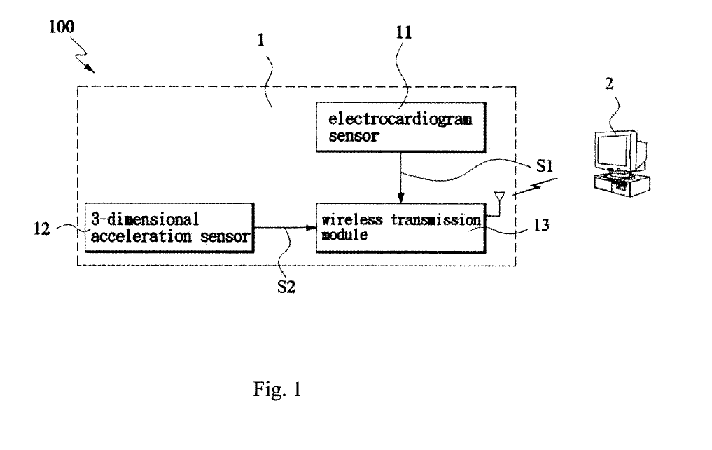

FIG. 1 is a block diagram of spontaneous exercise electrocardiogram system of the present invention. As shown in FIG. 1, the spontaneous exercise electrocardiogram system 100 comprises an electrocardiogram device 1 and an analyzing computer 2.

The electrocardiogram device 1 comprises an electrocardiogram sensor 11, a 3-dimensional acceleration sensor 12 and a wireless transmission module 13. The electrocardiogram sensor 11 is adapted to capture and record an electrocardiogram signal S1 of a user to obtain its electrocardiogram (ECG) data, and the 3-dimensional acceleration sensor 12 is adapted to capture an acceleration signal S2 of the user to obtain its acceleration variation. The signals captured by the electrocardiogram sensor 11 and 3-dimensional acceleration sensor 12, after signal amplification and A/D conversion processes, are transmitted to the analyzing computer 2 by the wireless transmission module 13.

While the analyzing computer 2 receives the ECG data from the electrocardiogram device 11 to obtain the parameters of heart rate and heart rate variability of the user. In addition, the analyzing computer 2 receives the user's acceleration signal S2 to determine a physical indicator of the user, such as physical activity (PA). Upon the user's heart rate and heart rate variability parameters are matched and analyzed, the user's spontaneous exercise electrocardiogram is accordingly established.

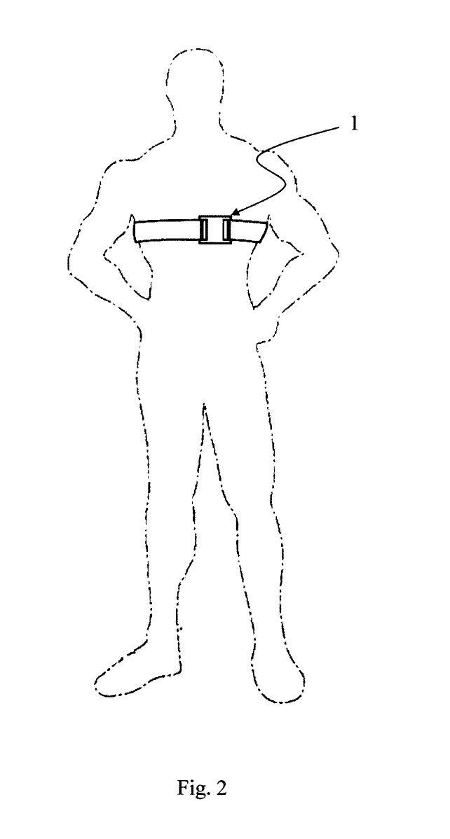

With reference to FIG. 2 which illustrates a belt type electrocardiogram device. However, the electrocardiogram device of the present invention is not limited to a belt type. As known by a skilled person, a neck-worn type, patch type, button type, or wrist-worn type 12-lead electrocardiogram device can be applied in the present invention, too. (Not shown in figure)

### PA (Physical Activity)

In the prior art, usually a predetermined threshold is set while measuring acceleration for a person by an acceleration sensor. There are different acceleration variations occurred in movements. When an acceleration variation exceeds the predetermined threshold (e.g. 0.025 g), it is recorded as a movement. During a predetermined period, the counts of movements are accumulated to represent the physical activity (PA) of the person (unit: count/minute).

### NPA (New Physical Activity)

Traditional acceleration sensors are accurate. However, they have an inevitable drawback that results of measuring physical activity by thresholds are in all-or-none principle and the exact amount of physical activity can not be measured. In this case, a vigorous or gentle movement is counted as one equal movement after analysis. On the contrary, a movement under the threshold is ignored, so that sensitivity of analysis is limited.

To solve the problems, a novel analysis has been developed. With a better strategy, all captured signals are processed without predetermination, so that all information will not be ignored. The 3-dimensional acceleration sensor has three recording channels, and the components of x-axis, y-axis and z-axis at each time-point are simultaneously recorded. Total amount A (unit=G) at each time-point is calculated by the formula defined as A=√x²+y²+z². The value contains the components of x, y and z axis, so that it is variable. Root mean square (RMS) is further applied to calculate to obtain the acceleration variation during a specified time segment (about 1 sec), and the acceleration variation can represent the physical activity.

More clearly, the method for recording physical activity by using RMS, is a new method of physical activity evaluation. The results according this method are defined as new physical activity (NPA), which can represent physical energy expenditure as traditional physical activity (PA).

### Example 1

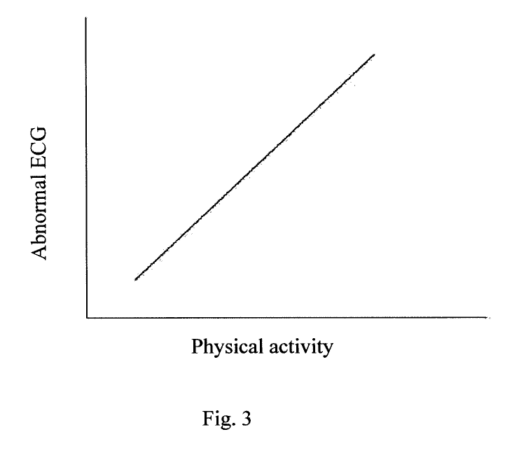

FIG. 3 illustrates relations between abnormal ECG and physical activity. It is also the primary principle the present invention based on. As shown in the diagram, the abnormal ECG is directly proportional to the physical activity (PA). Furthermore, physical indicators such as new physical activity (NPA) or other indicators derived from the acceleration signal sensed by the 3-dimensional acceleration sensor. The abnormal ECG represents cardiac abnormality including abnormal PQRST waves, heart rate and heart rate variability of electrocardiogram.

In the present invention, the computer is applied to automatically determine the instantaneous physical activity and retain instantaneous ECG analysis, so as to replace traditional treadmills. Users can keep their routine life normally without doing some specific movements at particular place (i.e. hospital). While the accumulated data is sufficient, relation between physical activity and abnormal ECG can be established, and the spontaneous exercise electrocardiogram is accordingly obtained.

In operation, firstly the electrocardiogram device is worn to simultaneously record ECG data and 3-axis acceleration signals. The signals captured by the 3-dimensional acceleration sensor can be transformed to an instantaneous physical activity through the analyzing computer. Then the heart rate and heart rate variability parameters are matched and analyzed, the user's spontaneous exercise electrocardiogram is accordingly established. On the other hand, the system of the present invention can be constantly executed for 24 hr, and by means of automatic detection, automatic identification and automatic wireless transmission, it is suitable for personal health care. Due to compact design of the device, it also meets the artistic requirement. For example, a neck-worn type, patch type, button type, wrist-worn type, or belt type 12-lead electrocardiogram device is available.

### Heartbeat Identification

The digitized ECG signals are processed as follow: At first, all peaks of the digitized ECG signals are detected by a spike detection algorithm to represent each heartbeat. Parameters such as amplitude and duration of all spikes are measured so that their means and standard deviations (SD) could be calculated as standard templates for following comparison. If an electrocardiogram signal, whose comparison result exceeds 3 standard deviations, it will be deemed a noise and be removed. Next, the interval between two adjacent peaks is measured to be the period of the heart beat. The means and standard deviations of all periods are calculated to confirm all periods of heart beats. If a period exceeds 3 standard deviations, the signal will be deemed a noise and be removed. Qualified signals will proceed further analysis.

### Frequency Domain Analysis

All qualified peaks are sampled at an appropriate frequency, e.g., 7.11 Hz. Fourier transform is adopted in spectrum analysis. In the first place, any linear drift of signal is eliminated to evade the interference from low-frequency band, and the Hamming computation is employed to prevent the mutual leakage between individual frequency components of the spectrum. After that, 288-second data (2048 points) is taken and fast Fourier transform is conducted so as to acquire heart rate power spectral density (HPSD), and the compensation with regard to any effects of sampling and Hamming computation is performed. The powers of the LF (0.04-0.15 Hz) and HF (0.15-0.4 Hz) bands of the heart rate power spectral density are quantified by integral, and the quantitative parameters like LF/HF or TP are figured out as well.

### Interpretation of Frequency Domain Analysis

This part is based on the method of applicant's previous publications (Kuo et al. 1999; Kuo et al. 1997; Yang et al. 2000; Yien et al. 1997), and the common view of some cardiologists in U.S. and Europe (anonymous, 1996). According to the results in the documents, HF and TP are cardiac parasympathetic indicators, and LF is sympathetic and parasympathetic indicator.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A spontaneous exercise electrocardiogram system, adapted to obtain and synchronously record a spontaneous exercise electrocardiogram of a user, and determine whether the user has cardiac abnormality, the system comprising:
an electrocardiogram device, comprising an electrocardiogram sensor for capturing and recording an electrocardiogram signal of the user to obtain an electrocardiogram (ECG) data, and a 3-dimensional acceleration sensor for capturing an acceleration signal of the user to obtain an acceleration variation; and
an analyzing computer, adapted to receive the ECG data from the electrocardiogram device to obtain the heart rate and heart rate variability of the user; and to receive the user's acceleration signal to determine a physical indicator of the user, wherein upon the user's heart rate and heart rate variability are matched and analyzed, the user's spontaneous exercise electrocardiogram can be established.

2. The spontaneous exercise electrocardiogram system as claimed in claim 1, wherein the electrocardiogram device is a neck-worn type, patch type, button type, wrist-worn type, or belt type 12-lead electrocardiogram device.

3. The spontaneous exercise electrocardiogram system as claimed in claim 1, wherein the electrocardiogram device further comprises a wireless transmission module adapted to transmit the signal of electrocardiogram sensor and 3-dimensional acceleration sensor to the analyzing computer.

4. The spontaneous exercise electrocardiogram system as claimed in claim 1, wherein the physical indicator is a PA (physical activity), NPA (new physical activity) or an indicator derived from the acceleration signal of the user.

5. The spontaneous exercise electrocardiogram system as claimed in claim 1, wherein the cardiac abnormality comprises abnormal PQRST waves, heart rate and heart rate variability of electrocardiogram.

6. The spontaneous exercise electrocardiogram system as claimed in claim 1, wherein the instantaneous physical indicator of the user is automatically obtained by the analyzing computer and matched with the instantaneous electrocardiogram.

7. The spontaneous exercise electrocardiogram system as claimed in claim 1, wherein the components of x-axis, y-axis and z-axis at each time-point of the user is sensed and cumulated to obtain the total acceleration value for each time-point, and the total acceleration value is calculated to obtain its root mean square (RMS) of acceleration variation to be the acceleration variation of the user.
